(19) United States
(12) Patent Application Publication
(10) Pub. No.: US 2008/0182990 A1
(43) Pub. Date: Jul. 31, 2008

(54) Process for the Preparation of Irinotecan Hydrochloride Trihydrate

(75) Inventors: B. Vishnukant, Raichur (IN); Prashant Purohit, Raiehur (IN); K. Paparao, Raiehur (IN); Veereshapa, Raiehur (IN)

Correspondence Address: LOWE HAUPTMAN HAM & BERNER, LLP, 1700 DIAGONAL ROAD, SUITE 300, ALEXANDRIA, VA, 22314, US

(73) Assignee: SHILPA MEDICARE LIMITED, Raichur (IN)

(21) Appl. No.: 11/573,397

(22) PCT Filed: Aug. 9, 2004
(86) PCT No.: PCT/IB04/02626

§ 371 Date: Sep. 14, 2007

(51) Int. Cl. C07D 471/22

(52) U.S. Cl. 546/48

(57) ABSTRACT

The invention relates to an improved process for the preparation of Irinotecan hydrochloride trihydrate of formula (4) of enhanced yield, purity by contacting 1-chlorocarbonyl-4-piperidinopiperidine hydrochloride with 7-ethyl-10-hydroxy-camptothecin [IRT-3 (synthetic)] to obtain crude Irinotecan which is subsequently purified by solvent treatment, obtaining purified irinotecan which is converted into irinotecan hydrochloride trihydrate and the invention also relates to a report of the compound 1-chlorocorbonyl-4-piperidinopiperidine hydrochloride of formula (1) and its process for preparation.

### TECHNICAL FIELD

The present invention relates to an improved process for the preparation of Irinotecan hydrochloride trihydrate of formula (4) from 7-ethyl-10-hydroxy-camptothecin of formula (2). The invention also relates to a report of compound 1-chlorocarbonyl-4-piperidinopiperidine hydrochloride of formula (1), its process for preparation and use in obtaining Irinotecan hydrochloride trihydrate.

### BACKGROUND AND PRIOR ART REFERENCES

Irinotecan of formula (3) and its hydrochloride trihydrate salt of formula (4) have been reported to be an antileukemic agent. In the prior art the hydrochloride salt has been obtained from semi-synthetically or synthetically prepared irinotecan.

Documents CZ 2002 2250, WO 9631513, U.S. Pat. No. 6,121,451, U.S. Pat. No. 6,252,079, U.S. Pat. No. 6,444,820, U.S. Pat. No. 6,723,729, WO 030 89413 and WO 9901456 describes the preparation of camptothecin derivatives including irinotecan.

Prior art processes describe preparation of Irinotecan hydrochloride trihydrate from camptothecin by obtaining 7-ethyl-10-hydroxy-camptothecin (3) as one of the intermediate and contacting with 1-chlorocarbonyl-4-piperidinopiperidine base (herein further referred to as IRT-4) to obtain crude Irinotecan which is purified by column chromatography and further converted into its hydrochloride trihydrate salt.

The present invention uses 7-ethyl-10-hydroxycamptothecin [herein further referred to as IRT-3 (synthetic)] as a starting material and contacting with 1-chlorocarbonyl-4-piperidinopiperidine hydrochloride (herein further referred to as IRT-4.HCl) to obtain crude irinotecan which is purified by adopting a simple process of solvent treatment and converting purified irinotecan into Irinotecan hydrochloride trihydrate having enhanced yield and purity.

Further the process of present invention obviates the step of column chromatography as a purification step enabling the present process economical and simple.

So far, 1-chlorocarbonyl-4-piperidinopiperidine hydrochloride and its use to obtain Irinotecan hydrochloride trihydrate has not been cited in the prior art. Now, the same has been used in the present invention which has led to surprising results of enhanced yield of Irinotecan hydrochloride trihydrate to two folds having improved purity level.

The above objectives could not have been achieved successfully but for using 7-ethyl-10-hydroxy-camptothecin (synthetic) and 1-chlorocarbonyl-4-peperidinopiperidine hydrochloride as starting material in the present invention. Thus, the use of 1-chlorocarbonyl-4-piperidinopiperidine hydrochloride in the preparation of Irinotecan hydrochloride trihydrate has led to surprising results establishing the novelty and inventiveness of the present invention. The result has been illustrated with examples and substantiated by the results in the present application (Reference Table I).

Also, there has been no report of the compound 1-chlorocarbonyl-4-peperidinopiperidine hydrochloride of formula (1) and its process of preparation in the prior art.

Further, the use of 1-chlorocarbonyl-4-peperidinoopiperidine hydrochloride having enhanced storage stability enables the process of present invention more consistent and easily operable.

In the present application the compound coded as IRT-3 (Semi-synthetic) refers to 7-ethyl-10-hydroxy-camptothecin obtained from camptothecin of natural origin and IRT-3 (Synthetic) refers to 7-ethyl-10-hydroxy-camptothecin available in the market.

### OBJECTS OF THE INVENTION

An object of the invention is to provide 1-chlorocarbonyl-4-piperidinopiperidine hydrochloride as one of the reactants for obtaining Irinotecan hydrochloride trihydrate.

Another object of the invention is to provide a process for the preparation of 1-chlorocarbonyl-4-piperidinopiperidine hydrochloride.

Yet another objective of the invention is to provide an improved process for the preparation of Irinotecan hydrochloride trihydrate.

Still another object of the invention is to provide a process which is economical and simple to perform.

Still yet another object of the invention is to provide a process which obviates the step of column chromatography purification.

An object of the invention is to provide 1-chlorocarbonyl-4 peperidopiperidine hydrochloride having enhanced storage stability.

Another object of the invention is to provide a process for preparing Irinotecan hydrochloride trihydrate with enhanced yield and purity.

### SUMMARY OF THE INVENTION

The invention relates to an improved process for the preparation of Irinotecan hydrochloride trihydrate of enhanced yield, purity by contacting with 1-chlorocarbonyl-4-piperidinopiperidine hydrochloride and 7-ethyl-10-hydroxy-camptothecin (synthetic) to obtain Irinotecan which is subsequently purified by solvent treatment and converted into Irinotecan hydrochloride trihydrate. The present invention also relates to a report of 1-chlorocorbonyl-4-piperidinopiperidine hydrochloride and its process for preparation

### BRIEF DESCRIPTION OF FIGURES AND TABLE

FIG. 1: HPLC Chromatogram of irinotecan (IRT5) obtained as per example 8.

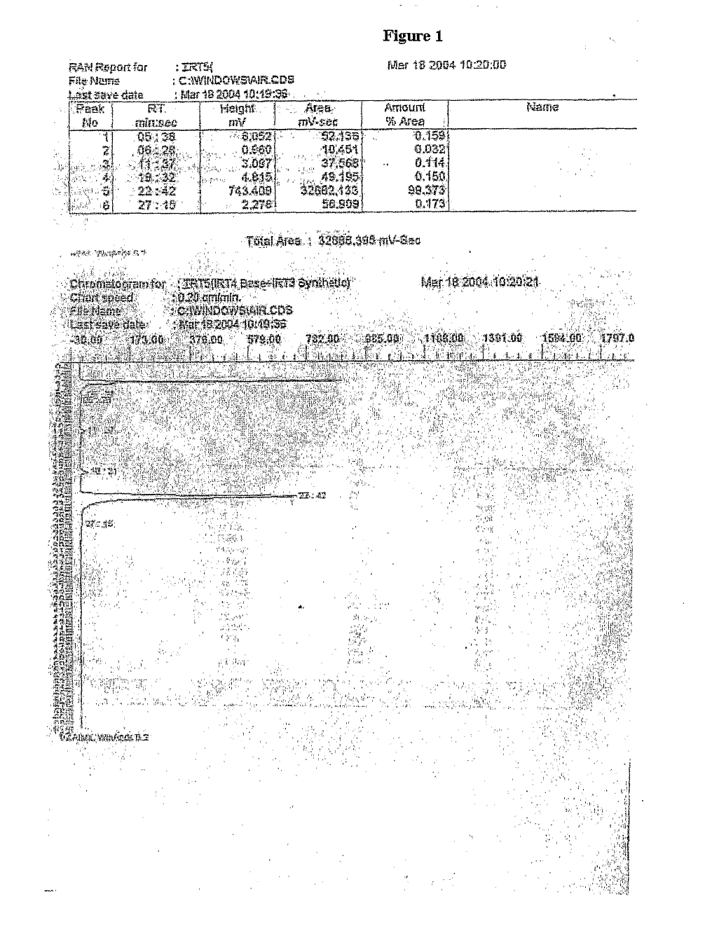

FIG. 2: HPLC Chromatogram of irinotecan hydrochloride trihydrate (IRT.HCl.3H20) obtained as per example 8.

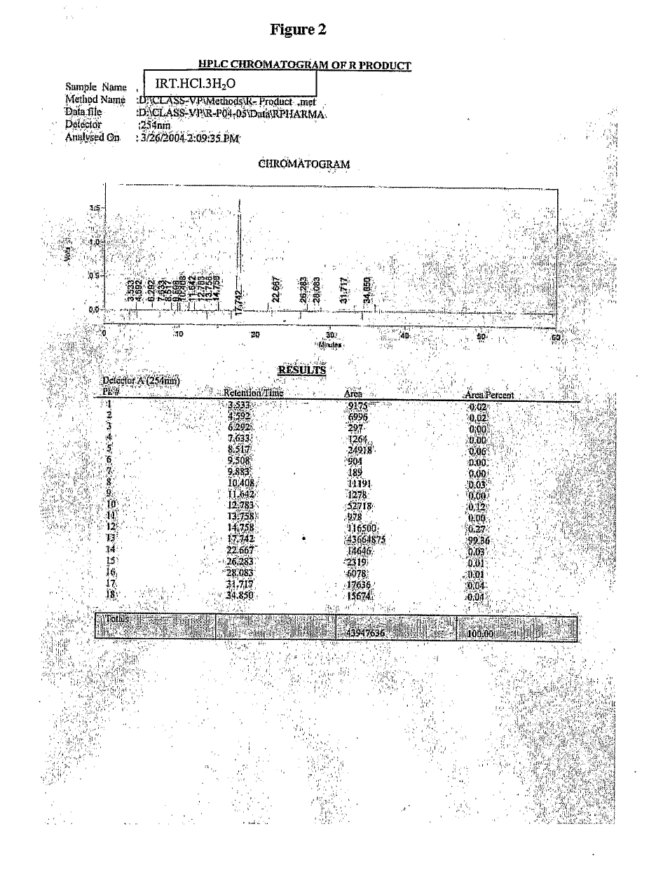

FIG. 3: HPLC Chromatogram of irinotecan (IRT5) obtained as per example 5.

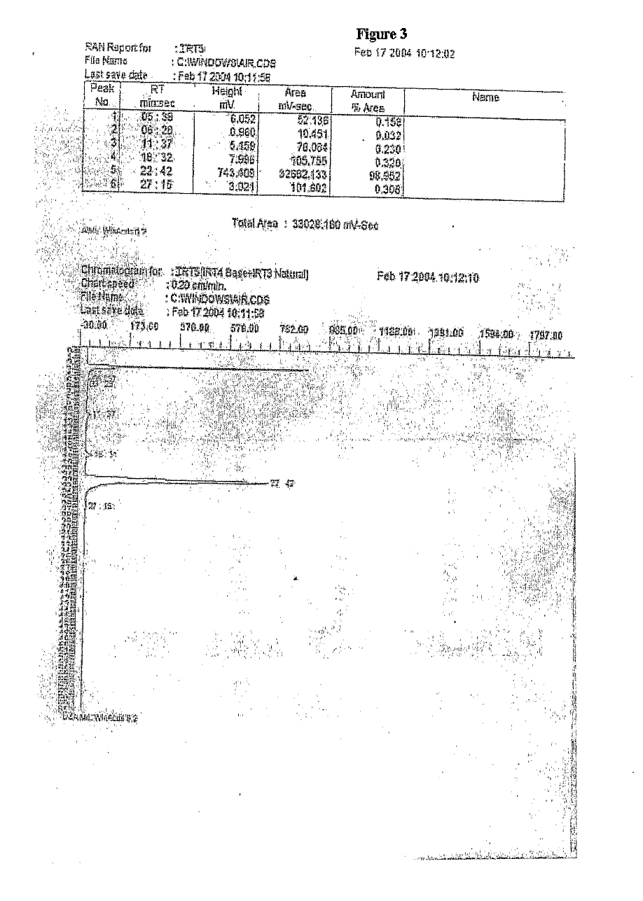

FIG. 4: HPLC Chromatogram of irinotecan hydrochloride trihydrate (IRT.HCl.3H20) obtained as per example 6.

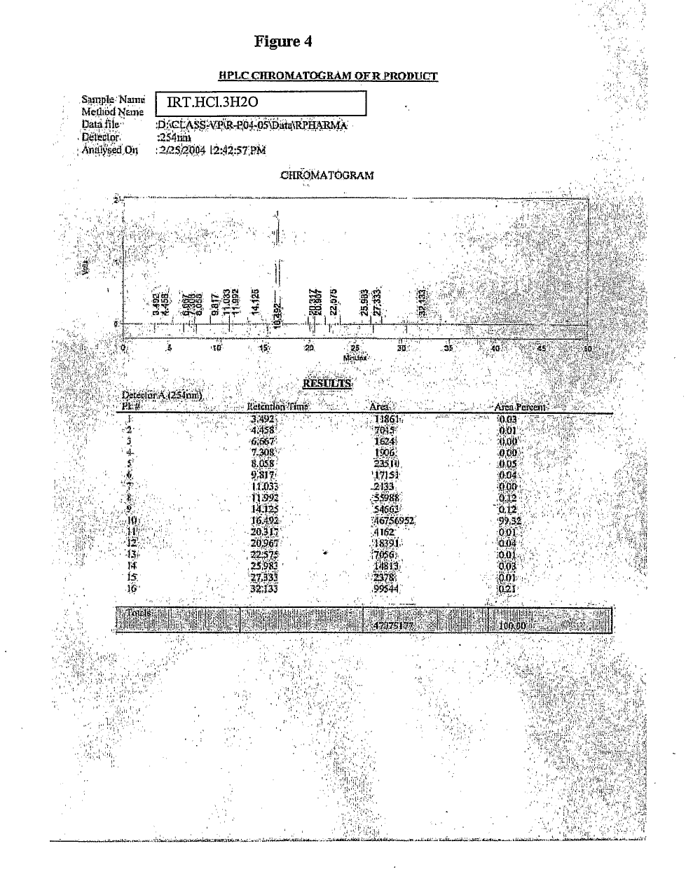

FIG. 5: HPLC Chromatogram of irinotecan (IRT5) obtained as per example 7.

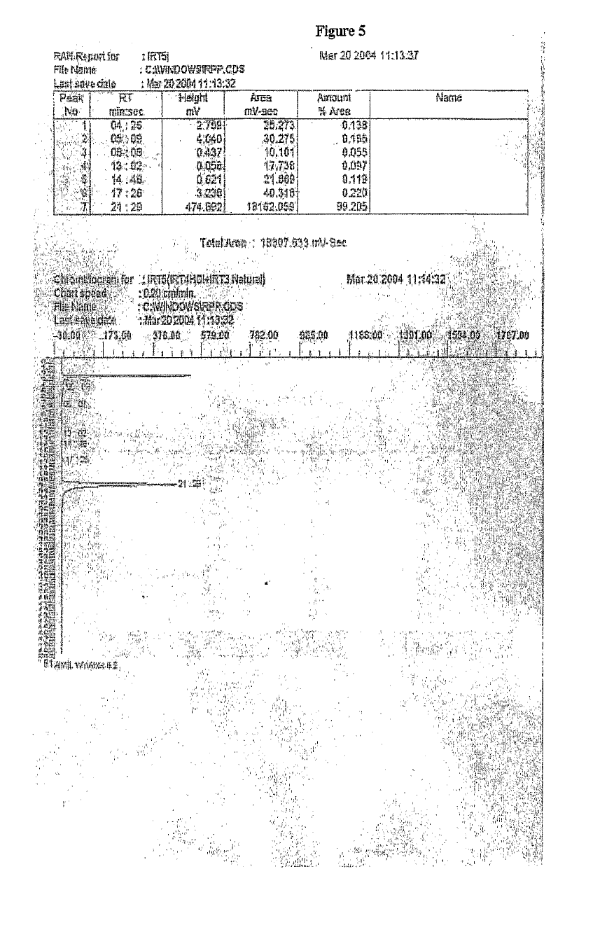

FIG. 6: HPLC Chromatogram of irinotecan hydrochloride trihydrate (IRT.HCl.3H20) obtained as per example 7.

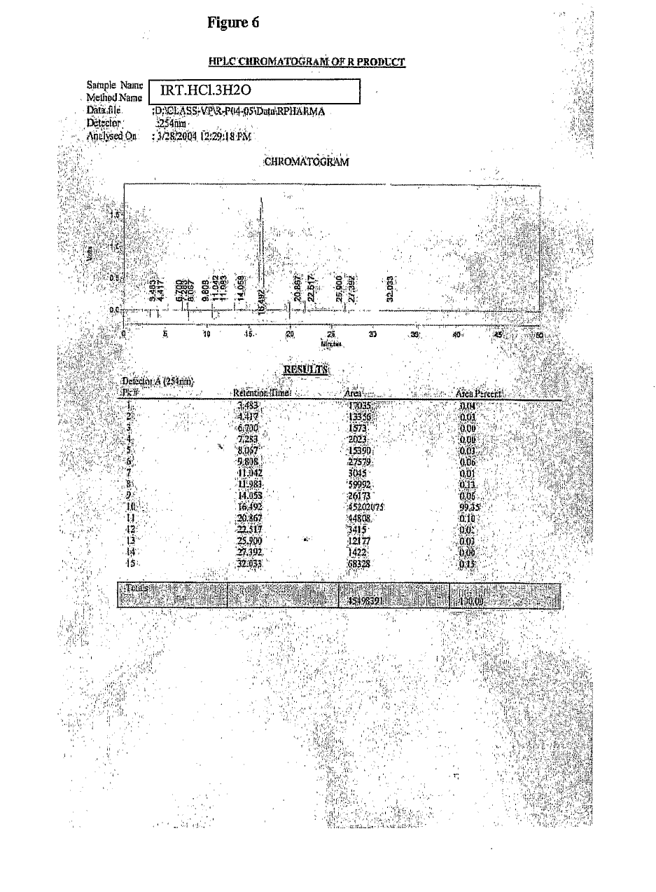

FIG. 7: HPLC Chromatogram of irinotecan (IRT5) obtained as per example 9.

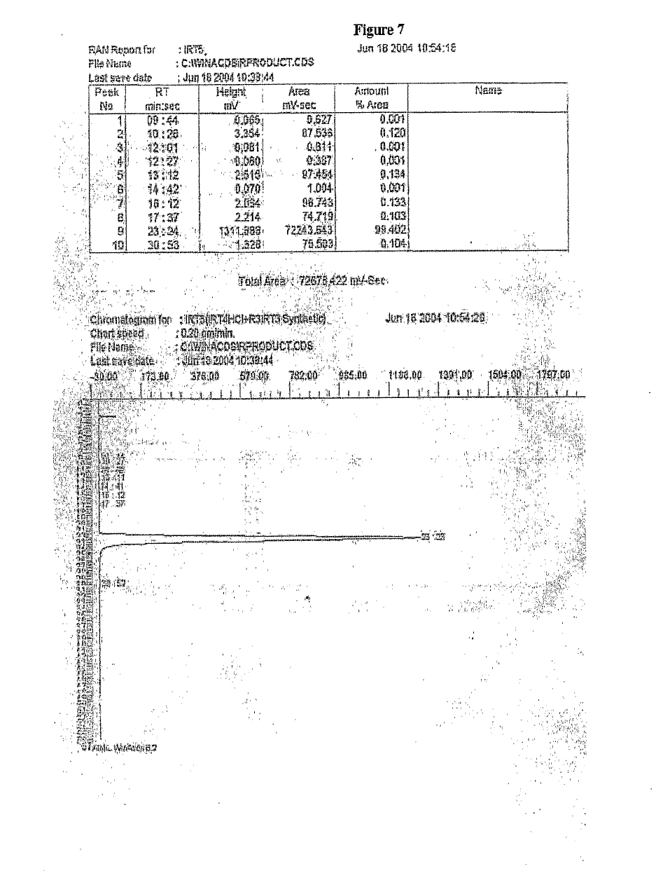

FIG. 8: HPLC Chromatogram of irinotecan hydrochloride trihydrate (IRT.HCl.3H20) obtained as per example 9.

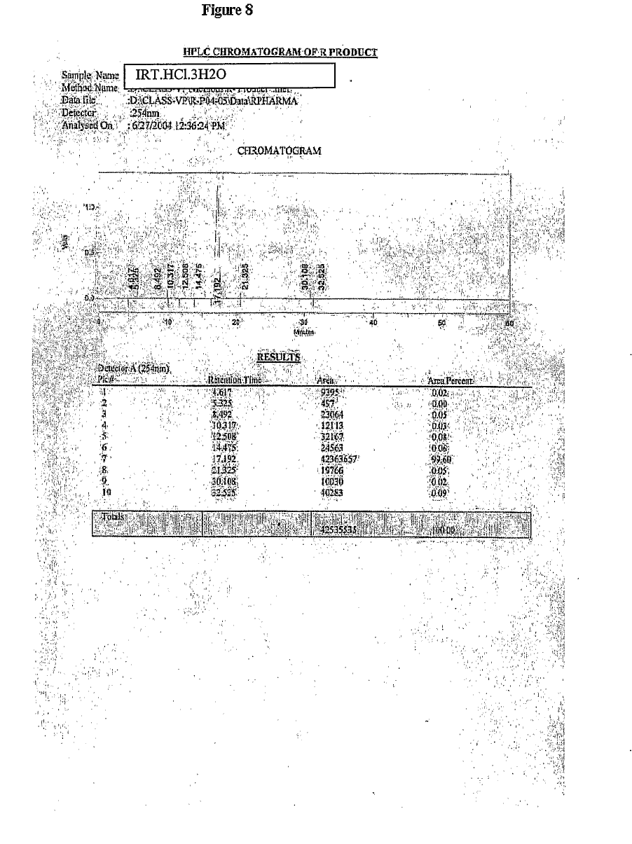

Table-1: Yield and % purity of irinotecan and irinotecan hydrochloride trihydrate obtained in examples 5 to 9.

### DETAILED DESCRIPTION OF THE INVENTION

In accordance, the present invention relates to an improved process for the preparation of Irinotecan hydrochloride trihydrate, the said process comprising steps of:
a) dissolving by stirring 7-ethyl-10-hydroxycamptothecin in pyridine at room temperature,
b) adding the solution of 1-chlorocarbonyl-4-peperidinopiperidine hydrochloride in pyridine to step (a) solution at room temperature, continued stirring the mixture for a period of 6 hours to 10 hours;
c) removing pyridine from step (b) mixture by distilling at a temperature below 60° C., preferably below 45° C. under reduced pressure to obtain a residue, cooling the residue to room temperature;
d) dissolving the residue of step (c) in aliphatic halogenated hydrocarbon solvent;
e) washing the solution of step (d) with an aqueous sodium bicarbonate followed by DM water three times,
f) separating the organic layer of step (e), distilling the organic solvent under reduced pressure to obtain an oily residue,
g) cooling the oily residue of step (f) to room temperature, adding alkane solvent stirring at room temperature to obtain a precipitate;
h) separating the precipitate of step (g), washing with alkane solvent, drying under reduced pressure at temperature ranging between 40° C.-50° C. preferably 45° C. to obtain crude irinotecan,
i) treating the crude irinotecan of step (h) with a mixture of dimithylformamide and alcohol for 8 hours to 14 hours filtering to obtain a residue of pure irinotecan;
j) preparing aqueous hydrochloric acid, adding residue of step (i), stirring for a period of 1 hour to 2 hours to obtain a solution;
k) charging carbon to step (j) solution, stirring for further 30 minutes, filter, collecting the filtrate, washing the filtrate with chloroform;
l) removing water partially from the washed filtrate of step (k) at a temperature ranging between 40° C. to 60° C. preferably below 45° C. under vacuum,
m) cooling the concentrated solution of step (I) to room temperature, then to 0° to 5° C. for a period of 2 hours to 14 hours, crystallizing Irinotecan hydrochloride trihydrate and
n) separating the product of step (m) and drying at a temperature ranging between 40° to 50° C. preferably below 45° C. under reduced pressure to obtain Irinotecan hydrochloride trihydrate of formula (4).
   The present process is depicted by the scheme as shown in next page

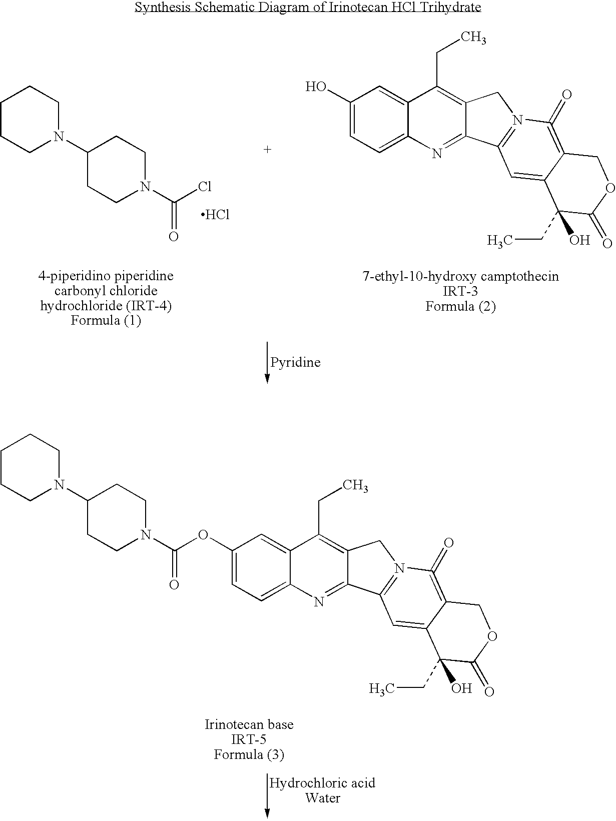

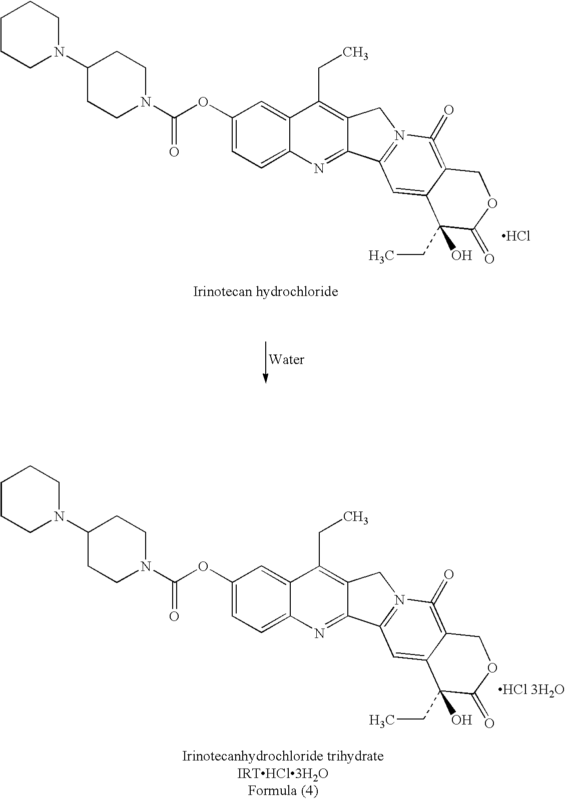

An embodiment of the invention provides the use of 1-chlorocarbonyl-4-piperidinopiperidine hydrochloride of formula (1) which is obtained by the said process comprising steps of;
i) preparing a solution of triphosgene by dissolving under stirring in aliphatic halogenated hydrocarbon solvent at room temperature;
ii) adding solution of step (i) to a solution of 4-piperidinopiperidine in aliphatic halogenated hydrocarbon solvent over a period of 2 hours to 6 hours at a temperature ranging between 5° C. to 10° C.;
iii) stirring the mixture of step (ii) for further 2 hours to 4 hours raising the temperature up to 30° C., maintaining for 6 hours to 8 hours;
iv) removing aliphatic halogenated hydrocarbon solvent completely from step (iii) mixture under vacuum at a temperature up to below 45° C.; cooling the residue to room temperature, adding alkane solvent, stirring, filtering the solid, drying, and
v) obtaining 1-chlorocarbonyl-4-piperidinopiperidine hydrochloride.

Another embodiment of the present invention provides the use of aliphatic halogenated hydrocarbon solvent preferably chloroform in the work up for obtaining crude irinotecan.

Yet another embodiment of the present invention provides the use of alkane solvent preferably n-hexane for precipitating the product crude irinotecan.

Still another embodiment provides the use of aliphatic halogenated hydrocarbon preferably chloroform for removing the impurities before crystallizing irinotecan hydrochloride trihydrate.

Sill yet another embodiment of the invention report the compound 1-chlorocorbonyl-4-piperidinopiperidine hydrochloride.

Another embodiment of the invention provides a process for preparing 1-chlorocarbonyl-4-piperidinopiperidine hydrochloride

Yet another embodiment of the invention provides the use of aliphatic halogenated hydrocarbon solvent preferably methylene dichloride in the preparation of 1-chlorocarbonyl-4-piperidinopiperidine hydrochloride

Still another embodiment of the invention provides the use of alkane solvent preferably n-hexane in the preparation of 1-chlorocarbonyl-4-piperidinopiperidine hydrochloride

Still yet another embodiment of the invention provides 1-chlorocarbonyl-4-piperidinopiperidine hydrochloride having enhanced storage stability

An embodiment of the invention provides the process for obtaining irinotecan hydrochloride trihydrate having enhanced yield by two folds.

Another embodiment of the present invention provides irinotecan hydrochloride trihydrate having the purity of up 99.60% with accompanying major known impurity up to 0.06% and major unknown impurity up to 0.09%.

Further embodiment of the invention provides a process to obtain irinotecan hydrochloride trihydrate of enhanced yield and purity.

The invention is illustrated with example and should not be construed to limit the scope of the present invention.

### EXAMPLES

### Example 1

### Preparation of 1-chlorocarbonyl-4-piperidinopiperidine Base (IRT-4)

Dissolving 4-piperidinoperidine (100 g) in benzene (1580 ml) under stirring for 15 to 30 minutes at room temperature, adding a solution of triphosgene (150 g) in benzene (660 ml) over a period of 1 to 3 hours at a temperature of 20°-25° C. Filtering the solid, washing it with benzene and drying, then dissolve the dried solid in chloroform (5900 ml) by stirring at room temperature for about 30 minutes. Charging aqueous sodium bicarbonate solution (400 ml), stirring and separating chloroform layer, washing the chloroform layer with water (1800 ml), separating chloroform layer and distilling off chloroform under vacuum at a temperature up to below 45° C. to obtain 1-chlorocorbonyl-4-piperidopiperidine base (60 g).

### Example-2

### Preparation of 1-chlorocarbonyl-4-piperidinopiperidine hydrochloride (IRT4.HCl)

Dissolving triphosgene (110 g) in dichloromethane (300 ml) with stirring for 15 to 30 minutes at room temperature, adding a solution of 4-piperidinopiperidine (100 g) in dichloromethane (300-ml) slowly over a period of 2 hour to 6 hours, maintaining the temperature between 5° to 10° C. Stirring the mixture keeping the temperature same for further period of 2 hours to 4 hours, raising the temperature up to 30° C., maintaining at this temperature for further period of 6 to 8 hours and distilling the dichloromethane completely at a temperature up to below 45° C. under vacuum. Cooling the residue to room temperature and adding n-hexane (200 ml), stirring filtering and drying to obtain 1-chlorocarbonyl-4-piperidinopiperidine hydrochloride (120 g)

### Example-3

### Preparation of 1-chlorocarbonyl-4-piperidinopiperidine hydrochloride(IRT-4.HCl)

Procedure followed is same as in Example 2 except the solvent used is chloroform.

### Example-4

### Preparation of 7-ethyl-10-hydroxycamptothecin (IRT-3, semi-synthetic) from Camptothecin

Step-a: Camptothecin (100 g) is taken in DM water (2000 ml), adding slowly concentrated sulfuric acid (1100 ml) at a temperature ranging between 45°-55° C., further maintaining the temperature around 60° C. for a period 2 hours to 4 hours, cooling the mixture to about minus 3° C., adding propionaldehyde (48 ml) and maintaining the temperature between 0° to 3° C. Adding ferrous sulphate, hydrogen peroxide (130 ml), raising the temperature up to about 30° C. and maintaining for about 2 hours. Charging this mixture to a solution of aqueous sodium sulphate, extracting with chloroform (3×5 l), followed by washing the chloroform layer with water, separating chloroform layer and removing completely chloroform under vacuum up to below 45° C., obtaining a residue, treating it with dimethyl formamide (3900 ml) at a temperature ranging between 80°-100° C., cooling to 0° to 5° C., filtering, washing with methanol, drying to obtain the required product of 7-ethylcamptothecin (68 g).
Step-b: Charging platinum oxide (14 g) in glacial acetic acid (600 ml) flushing with hydrogen and heating to 50°-60° C. under hydrogen atmosphere around 60 psi for about 2 hours. Cooling to room temperature and adding 7-ethylcamptothecin (70 g) in DMSO (5 ml) and hydrogenating at a temperature of about 60° C. for a period of about 8 hours. Filtering the mixture, collecting the filtrate and adding DM water (300 ml) to it under nitrogen atmosphere, adding iodobenzene diacetate (168 g) in three lots at room temperature, stirring for further 3 hours, adding aqueous sodium acetate solution (1600 ml), stirring for an hour, filtering, washing to obtain wet 7-ethyl-10-hydroxycamptothecin, [280 g];
Step-c: Charging IRT-2 (280 g) to dimethylformaide (1400 ml) heating to 90°-100° C. for a period of about 30 minutes. Cooling to 0° to 5° C., filtering solid, washing with methanol, drying under vacuum at about 60°-80° C. to obtain purified 7-ethyl-10-hydroxy camptotheticin [IRT-3(semisynthetic); 48 g].

### Example-5

### Preparation of Irinotecan (IRT-5)

Dissolving 7-ethyl-10-hydroxycamptothecin (50 g) obtained in example 4(c) in pyridine (200 ml) under stirring at room temperature. Adding to it a solution of 1-chlorocarbonyl-4-piperidino-piperidine base (90 g) in pyridine (2000 ml) and stirring for further 6 hours to 12 hours. Distilling off pyridine completely under vacuum at a temperature preferably below 45° C., cool the residue to room temperature, dissolving in chloroform (2500 ml), washing the chloroform solution with an aqueous sodium bicarbonate, followed by water. Separating chloroform layer, removing chloroform completely under vacuum, adding n-hexane filtering, drying the solid, purifying by column chromatography to obtain purified Irinotecan (IRT-5, 30 g)

### Example-6

### Preparation of Irinotecan Hydrochloride Trihydrate (IRT.HCl.3H2O)

Charging IRT-5 (30 g) obtained in example 5 onto a mixture of DM water (1300 ml) and concentrated hydrochloride acid (28 ml), stirring for 1 to 4 hour to dissolve completely, washing the solution thus obtained with chloroform, collecting aqueous solution. Treating the aqueous solution with carbon under stirring, filtering, collecting filtrate and distilling partially water from washed filtrate under vacuum at a temperature below 45° C., cooling to 0° to 5° C. for a period of 10 hours to 12 hours, filter the solid obtained, drying under vacuum below 45° C. to obtain Irinotecan hydrochloride trihydrate (22.5 g).

### Example-7

7-ethyl-10-hydroxycampothecin (semi-synthetic) obtained in example 4 (c) and 1-chlorocarbonyl-4-piperidinopiperidine hydrochloride (90 g) are used as reactants. Following the procedure described in examples (5) and (6) irinotecan (32 g) and irinotecan hydrochloride trihydrate (30 g) respectively are obtained.

### Example-8

7-ethyl-10-hydroxycamptothecin [50 g; IRT-3(synthetic)] and 1-chlorocarbonyl-4-piperidinopiperidine (90 g) are used as reactants. Following the procedure of example (5) and obtaining crude irinotecan, purifying crude irinotecan by treatment with dimethyl formamide-alcohol mixture to obtain pure irinotecan (34 g). Irinotecan hydrochloride trihydrate (25 g) is prepared as per example (6) above.

### Example-9

7-ethyl-10-hydroxy camptothecin (50 g; synthetic) and 1-chlorocarbonyl-4-piperidinopiperidine hydrochloride (90 g) are used as reactants. By following the procedure of examples (8) and (6) irinotecan (80 g) and irinotecan hydrochloride trihydrate (60 g) are obtained respectively.

### Main Advantages of the Invention;

1) Provides enhanced yield of Irinotecan hydrochloride trihydrate.
2) Provides enhanced purity of Irinotecon hydrochloride trihydrate.
3) Provides the use of one of the reactant 1-chlorocarbonyl-4-piperidinopiperidine hydrochloride with enhanced storage stability.
4) Process is simple and economical.

TABLE 1
Yield and % purity of irinotecan and irinotecan hydrochloride
trihydrate obtained in examples 5 to 9.

| | | | | | Major | |
|---|---|---|---|---|---|---|
| | | | | | known | Major |
| | | | Yield | | Impurity | unknown |
| S. No. | Reference | Product | (g) | % Purity | (%) | Impurity (%) |
| 1. | Example 5 | Irinotecan | 30.00 | 99.95 | 0.32 | 0.30 |
| | Example 6 | Irinotecan hydrochloride | 22.50 | 99.32 | 0.12 | 0.12 |
| | | trihydrate | | | | |
| 2 | Example 7 | Irinotecan | 32.00 | 99.20 | 0.22 | 0.16 |
| | | Irinotecan hydrochloride | 30.00 | 99.35 | 0.06 | 0.15 |
| | | trihydrate | | | | |
| 3 | Example 8 | Irinotecan | 34.00 | 99.37 | 0.15 | 0.17 |
| | | Irinotecan hydrochloride | 25.00 | 99.36 | 0.27 | 0.12 |
| | | trihydrate | | | | |
| 4. | Example 9 | Irinotecan | 50.00 | 99.40 | 0.10 | 0.13 |
| | | Irinotecan hydrochloride | 60.00 | 99.60 | 0.06 | 0.09 |
| | | trihydrate | | | | |

Above results refers to yield and purity of irinotecan and irinotecan hydrochloride trihydrate from 50 grms of 7-ethyl-10-hydroxy camptothecin (semisynthetic and synthetic) respectively.

1-16. (canceled)

17. An improved process for the preparation of irinotecan hydrochloride trihydrate the said process comprising steps of:
a) dissolving by stirring 7-ethyl-10-hydroxycamptothecin of formula (2) in pyridine at room temperature;
b) adding solution of i-chlorocarbonyl-4-piperidinopiperidine hydrochloride of formula (1) in pyridine to step (a) solution at room temperature, continued stirring the mixture for a period of 6 to 10 hours;
c) removing pyridine from step (b) mixture by distilling at a temperature below 60° C., preferably below 45° C. under reduced pressure to obtain a residue, cooling the residue to room temperature;
d) dissolving the residue of step (c) in an aliphatic halogenated hydrocarbon solvent;
e) washing the solution of step (d) with an aqueous sodium bicarbonate, followed by DM water three times,
f) separating the organic layer, distilling the organic layer under reduced pressure to obtain an oily residue,
g) cooling the oily residue of step (f) to room temperature, adding an alkane solvent, stirring at room temperature to obtain a precipitate;
h) separating the precipitate of step (g), washing with the alkane solvent, drying under reduced pressure at a temperature ranging between 40° C. to 50° C. preferably 45° C. to obtain crude irinotecan,
i) treating the crude irinotecan of step (h) with a mixture of dimithylformamide and alcohol for 8 to 14 hours, filtering to obtain a residue of pure irinotecan of formula (3);
j) preparing aqueous hydrochloric acid, adding residue of step (i) and stirring for a period of 1 to 2 hours to obtain a solution;
k) charging carbon to step (j) solution, stirring for further 30 minutes, filtering, collecting the filtrate and washing the filtrate with aliphatic halogenated hydrocarbon solvent;
l) removing water partially from the washed filtrate of step (k) at a temperature ranging between 40° C. to 60° C. preferably below 45° C. under vacuum;
m) cooling the concentrated solution of step (I) to room temperature, then to 0° to 5° C for a period of 2 hours to 14 hours, crystallizing Irinotecan hydrochloride trihydrate; and
n) separating the product of step (m) and drying at a temperature ranging between 40° to 50° C. preferably below 45° C. under reduced pressure to obtain Irinotecan hydrochloride trihydrate of formula (4).

18. A process of claim 17, wherein in step (b) i-chlorocarbonyM-piperidinopiperidine used is obtained by the said process comprising steps of;
i) preparing a solution of triphosgene by dissolving under stirring in aliphatic halogenated hydrocarbon solvent at room temperature;
ii) adding solution of step (i) to a solution of 4-piperidinopiperidine in aliphatic halogenated hydrocarbon solvent over a period of 2 to 6 hours at a temperature ranging between 5° C. to 10° C.;
iii) stirring the mixture of step (ii) for further 2 to 4 hours, raising the temperature up to 30° C, maintaining for 6 hours to 8 hours;
iv) removing aliphatic halogenated hydrocarbon solvent completely from step (iii) mixture under vacuum at a temperature up to below 45° C.; cooling the residue to room temperature, adding alkane solvent, stirring, filtering the solid, drying; and
v) obtaining 1-chlorocarbonyl-4-piperidinopiperidine hydrochloride.

19. A process of claim 17, wherein in step (d) the aliphatic halogenated hydrocarbon solvent used, is selected from a group consisting of dichloromethane, dichloroethane and chloroform.

20. A process of claim 19, wherein the preferred solvent is chloroform.

21. A process of claim 17, wherein in step (g), the alkane solvent used, is selected from a group consisting of n-pentane, n-hexane and n-heptane.

22. A process of claim 21, wherein the preferred solvent is n-hexane.

23. A process of claim 17, wherein in step (k) the aliphatic halogenated hydrocarbon solvent used is selected from a group consisting of dichloromethane, dichloroethane and chloroform.

24. A process of claim 23, wherein the preferred solvent is chloroform.

25. A process of claim 18, wherein in step (i) the aliphatic halogenated solvent used is selected from a group consisting of carbon tetrachloride, chloroform, methylene dichloride and ethylene dichloride.

26. A process of claim 25, wherein the preferred solvent is methylene dichloride.

27. A process of claim 18, wherein in step (iv) the alkane solvent used is selected from a group consisting of n-pentane, n-hexane and n-heptane.

28. A process of claim 17, wherein the preferred solvent used is n-hexane.
